(19) United States
(12) Patent Application Publication
(10) Pub. No.: US 2026/0207891 A1
(43) Pub. Date: Jul. 23, 2026

(54) CATHETER

(71) Applicant: PIOLAX MEDICAL DEVICES, INC., Kanagawa (JP)

(72) Inventor: Masanori KITAGAWA, Yokohama-shi, Kanagawa (JP)

(73) Assignee: PIOLAX MEDICAL DEVICES, INC., Kanagawa (JP)

(21) Appl. No.: 19/142,966

(22) PCT Filed: Dec. 25, 2023
(86) PCT No.: PCT/JP2023/046448

§ 371 Date: Jun. 25, 2025

(30) Foreign Application Priority Data
Dec. 28, 2022 (JP) 2022-211066

(51) Int. Cl. A61M 25/00

(52) U.S. Cl. CPC A61M 25/005; A61M 25/0045; A61M 2025/0059

(57) ABSTRACT

This catheter has a tube including a braided reinforcement member. The reinforcement member includes: a first spiral section formed by winding first wire materials at prescribed intervals in a first spiral direction; and a second spiral section formed by winding a second wire material in a second spiral direction different from the first spiral direction. The second spiral section has parallel contact portions in which two second wire materials are arranged in parallel so as to contact each other. Parallel contact portions are formed by being wound at prescribed intervals.

### TECHNICAL FIELD

The present invention relates to a catheter used for injecting an anticancer drug or the like or inserting a stent or the like into, for example, a tubular organ of the human body such as a blood vessel, a bile duct, a pancreatic duct, a ureter, or a trachea.

### BACKGROUND ART

In the related art, a catheter is inserted into a tubular organ such as a blood vessel, a ureter, a bile duct, or a trachea or into a human tissue such as a body cavity to inject a contrast medium, an anticancer drug, a nutrient, or the like through the catheter or to insert a stent, a vaso-occlusive device, or the like through the catheter in combination with a guide wire or the like.

This catheter is a tube made of a predetermined synthetic resin material, and may include a reinforcement member to improve rigidity, kink resistance, or the like.

For example, Patent Literature 1 below describes a catheter including a shaft where an inner cavity communicating with a range from a tip end to a base end is formed In this shaft of the catheter, a reinforcement layer formed of a braid where thin wires are interwoven is provided in a range from a base end portion to a tip end portion, and the braid is configured by disposing a first helical portion that is made from one or more wires wound in a first helix direction and a second helical portion that is made from one or more wires wound in a second helix direction different from the first helix direction to intersect with each other.

In addition, the wires forming the first helical portion and the wires forming the second helical portion are disposed at predetermined intervals, respectively, and the interval between the wires forming the first helical portion is wider than the interval between the wires forming the second helical portion (refer to FIG. 4 of Patent Literature 1).

### CITATION LIST

### Patent Literature

Patent Literature 1: JP2012-29872A

### SUMMARY OF INVENTION

### Technical Problem

In the catheter described in above Patent Literature 1, the first helical portion and the second helical portion are wound and formed at the intervals between the wires, respectively, in the entire region in an axial direction (range from the base end portion to the tip end portion). Therefore, the flexibility may deteriorate.

That is, the wires forming both of the helical portions are disposed at the intervals, respectively. Therefore, the number of positions where the wires intersect with each other in both of the helical portions increases, and a region surrounded by the intersection portions is likely to be narrowed. Thus, the kink resistance as the entire catheter is relatively high, but the flexibility tends to deteriorate.

Accordingly, an object of the present invention is to provide a catheter where flexibility can be improved while maintaining kink resistance.

### Solution to Problem

In order to achieve the above-described object, according to the present invention, there is provided a catheter including a tube that includes a reinforcement member made from a braid, in which the reinforcement member includes a first helical portion formed by winding first wires at a predetermined interval in a first helix direction, and a second helical portion formed by winding second wires in a second helix direction different from the first helix direction, and the second helical portion includes parallel contact portions each of where at least two of the second wires are disposed in parallel to be in contact with each other, and the parallel contact portions are wound and formed at a predetermined interval.

### Advantageous Effects of Invention

According to the present invention, the second helical portion includes the parallel contact portion where at least two second wires are disposed in parallel to be in contact with each other, and the parallel contact portions are wound and formed at the predetermined interval. Therefore, while maintaining the kink resistance of the parallel contact portion, the flexibility can be improved with the interval between the parallel contact portions.

### BRIEF DESCRIPTION OF DRAWINGS

FIG. 1 is an enlarged cross-sectional view of a main portion illustrating one embodiment of a catheter according to the present invention

FIG. 2 is a cross-sectional view taken along arrow A-A of FIG. 1.

FIG. 3 is an enlarged view illustrating the main portion in the catheter according to the present invention.

FIG. 4 is a diagram illustrating comparison between a parallel contact portion made of two fine wires and one thick wire in the catheter.

FIG. 5 is a schematic diagram illustrating a flexibility test of Example and Comparative Example.

FIG. 6 is a diagram illustrating test results of the flexibility test.

FIG. 7 is a chart illustrating test results of a kink resistance test of Example and Comparative Example.

### DESCRIPTION OF EMBODIMENTS

### One Embodiment of Catheter

Hereinafter, one embodiment of a catheter according to the present invention will be described with reference to the drawings.

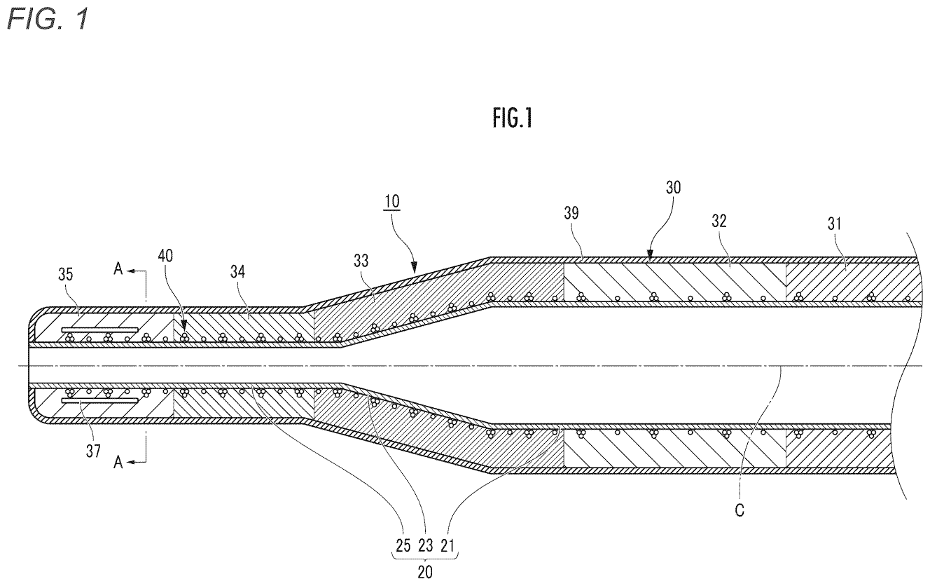

As illustrated in FIG. 1, a catheter 10 according to the present embodiment includes a tube that includes a reinforcement member 40 made from a braid. The tube has an inner cavity therein.

In the present embodiment, the tube forming the catheter 10 includes an inner layer 20 and an outer layer 30 that is disposed outside the inner layer 20.

In each of the members such as the catheter 10, the tube, the inner layer 20, the outer layer 30, the reinforcement member 40, a first helical portion 50, or a second helical portion 60, “tip end portion”, “tip end”, and “tip end side” refer to a distal end portion, a distal end, and a distal end side farthest from the hand side of a catheter manipulator, and “base end portion”, “base end”, and “base end side” refer to a proximal end portion, a proximal end, and a proximal end side closest to the hand side of the catheter manipulator In addition, as illustrated in FIG. 1, an axial center of the catheter 10 will be referred to as “axial center C”, and a direction along the axial center C will be referred to as an axial direction (the same also applies to the axial direction in each of the members).

As illustrated in FIG. 1, the inner layer 20 according to the present embodiment is configured with a base portion 21 having a fixed diameter that extends in a predetermined length, a tapered portion 23 that extends to be gradually tapered from a tip end of the base portion 21 toward a foremost tip end of the inner layer 20, and a tip end portion 25 that extends in a predetermined length from a tip end of the tapered portion 23 to have a fixed diameter less than an outer diameter of the base portion 21.

For the inner layer 20, for example, a fluorine-based resin such as polytetrafluoroethylene (PTFE), a perfluoroalkoxy resin (PFA), a tetrafluoroethylene-hexafluoropropylene copolymer (FEP), or a tetrafluoroethylene-ethylene copolymer (ETFE), polyurethane, a nylon elastomer, polyether block amide, polyethylene, polyvinyl chloride, vinyl acetate, a UV curable resin, or a resin used for an adhesive (an acrylate resin, a urethane adhesive, an epoxy adhesive, or a silicone adhesive) can be adopted. The inner layer 20 according to the present embodiment is formed of polytetrafluoroethylene (PTFE).

On the other hand, in the outer layer 30, a first layer 31, a second layer 32, a third layer 33, a fourth layer 34, and a fifth layer 35 are continuously provided in the axial direction from the base end side to the tip end side of the tube.

Specifically, the first layer 31 is disposed in a predetermined range of the base portion 21 of the inner layer 20. In addition, the second layer 32 is disposed on the tip end side with respect to a position of the base portion 21 of the inner layer 20 where the first layer 31 is disposed. Further, the third layer 33 is disposed in a range from a position on the tip end side with respect to a position of the base portion 21 of the inner layer 20 where the second layer 32 is disposed to the base end side of the tip end portion 25 through the tapered portion 23.

In addition, the fourth layer 34 is disposed in a predetermined range from a position on the tip end side with respect to a position of the tip end portion 25 of the inner layer 20 where the third layer 33 is disposed. Further, the fifth layer 35 is disposed on the tip end side with respect to a position of the tip end portion 25 of the inner layer 20 where the fourth layer 34 is disposed.

Further, for each of the layers 31 to 35 forming the outer layer 30, for example, polyurethane, polyester, a nylon elastomer, polyether block amide, polyethylene, polyvinyl chloride, vinyl acetate, a UV curable resin, a resin used for an adhesive (an acrylate resin, a urethane adhesive, an epoxy adhesive, or a silicone adhesive), or a fluorine-based resin such as polytetrafluoroethylene (PTFE), a perfluoroalkoxy resin (PFA), a tetrafluoroethylene-hexafluoropropylene copolymer (FEP), or a tetrafluoroethylene-ethylene copolymer (ETFE) can be adopted.

In addition, from the viewpoint of ensuring operability or torque transmissibility, the hardnesses of the layers 31 to 35 are set to gradually decrease in order from the highest hardness that is the hardness of the first layer 31 to the hardness of the fifth layer 35.

Further, as illustrated in FIG. 1, in the fifth layer 35 disposed on the tip end portion side of the outer layer 30, a radiopaque marker 37 that is formed of, for example, one of radiopaque metals made of W, Pt, Ti, Pd, Rh, Au, Ag, Bi, Ta, Ir, and alloys thereof is buried.

In addition, an outer periphery of the outer layer 30 is covered with a hydrophilic resin film 39 that is made of, for example, polyvinyl pyrrolidone, polyethylene glycol, or methyl vinyl ether-maleic anhydride copolymers.

Next, the reinforcement member 40 will be described in detail.

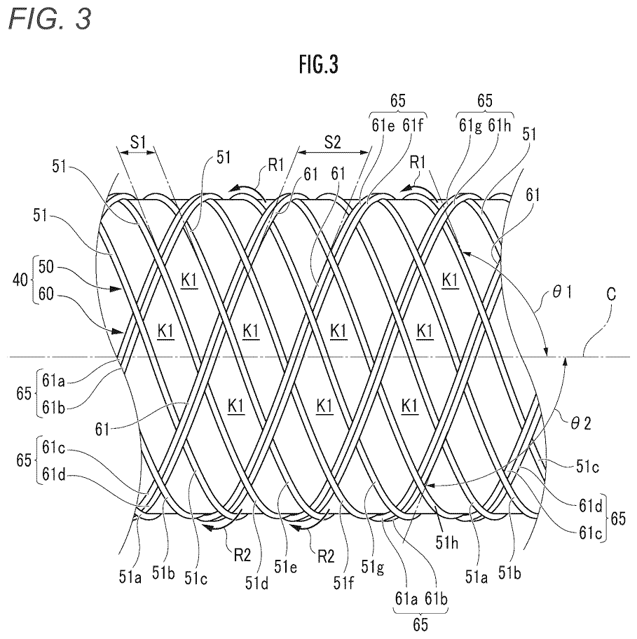

As illustrated in FIG. 3, this reinforcement member 40 includes: the first helical portion 50 formed by winding first wires 51 at a predetermined interval in a first helix direction; and the second helical portion 60 formed by winding second wires 61 in a second helix direction different from the first helix direction (the second helical portion 60 where the second wires 61 are wound in the second helix direction different from the first helix direction and are disposed to intersect with the first wires 51 of the first helical portion 50). In the present embodiment, both of the first wires 51 and the second wires 61 are round wires having a circular shape in cross-section.

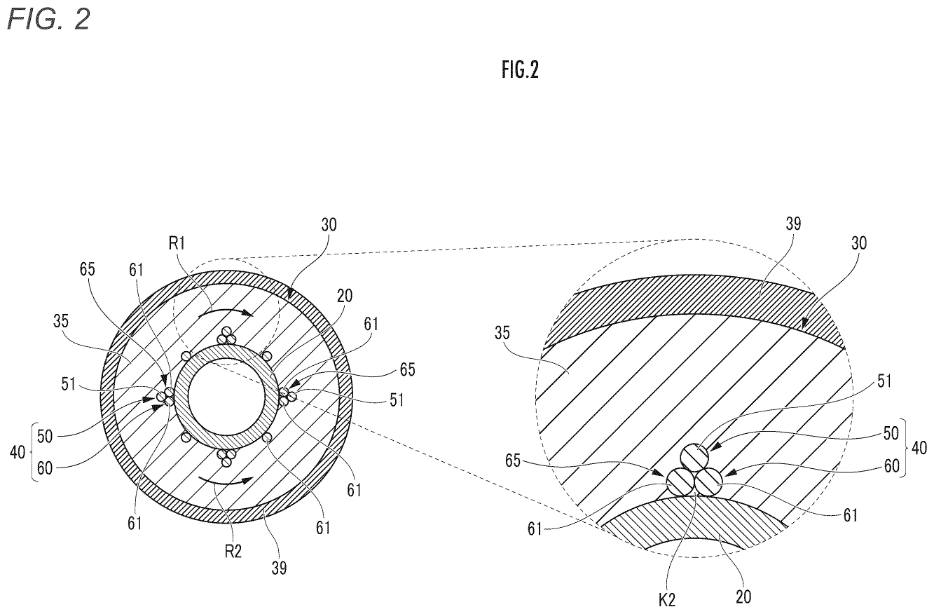

FIG. 2 is a cross-sectional view taken along arrow A-A of FIG. 1, that is, a cross-sectional view taken at a predetermined position on the tip end portion side of the catheter 10 when seen from the base end side toward the tip end side of the catheter 10. In addition, for convenience of description, FIG. 3 illustrates a state where the outer layer 30 is excluded. Further, in FIG. 3, the right side of the drawing is the base end side of the catheter 10, and the left side of the drawing is the tip end side of the catheter 10.

In addition, the above-described first helix direction RI (hereinafter, also simply referred to as “first helix direction RI”) is defined by an inclination angle θ1 of the first wires 51 with respect to the axial center C of the catheter 10 and a winding direction of the first wires 51. On the other hand, the above-described second helix direction R2 (hereinafter, also simply referred to as “second helix direction R2”) is defined by an inclination angle θ2 of the second wires 61 with respect to the axial center C of the catheter 10 and a winding direction of the second wires 61.

That is, in the first helical portion 50, while being inclined at the predetermined angle θ1 with respect to the axial center C of the catheter 10 as illustrated in FIG. 3, the first wires 51 are belically wound clockwise (clockwise) and formed as illustrated in FIG. 2 when the reinforcement member 40 is seen from the base end side to the tip end side of the catheter 10.

In addition, the first wires 51 and 51 are disposed at the predetermined interval S1 (refer to FIG. 3) in the axial direction of the first helical portion 50. This interval S1 on the base end side is wider than that on the tip end side of the first helical portion 50. For convenience of description, FIG. 3 illustrates an intermediate portion of the first helical portion 50. In this intermediate portion, the interval S1 between the first wires 51 and 51 is substantially fixed. The interval S1 on the base end side (in FIG. 3, the right side) with respect to the intermediate portion illustrated in FIG. 3 is wider than the interval S1 on the tip end side (in FIG. 3, the left side) with respect to the intermediate portion illustrated in FIG. 3.

The interval S1 is a length between a tangent line in one edge (in FIG. 3, the right edge) of one predetermined first wire 51 in a width direction and a tangent line in another edge (in FIG. 3, the left edge) of another first wire 51 in the width direction that is disposed adjacent to the predetermined first wire 51 in the axial direction of the first helical portion 50.

In addition, the interval SI between the first wires 51 and 51 of the first helical portion 50 is preferably 0.03 to 0.25 mm and more preferably 0.06 to 0.20 mm.

Further, the first helical portion 50 according to the present embodiment is made from eight first wires 51a, 51b, 51c, 51d, 51e, 51f, 51g, and 51h, and the first wires 51a, 51b, 51c, 51d, 51e, 51f, 51g, and 51h are wound and formed while maintaining the predetermined interval S1 in the axial direction of the first helical portion 50.

Here, the first wires 51a, 51b, 51c, 51d, 51e, 51f, 51g, and 51h are sequentially disposed from the tip end side to the base end side of the first helical portion 50. The first wire 51a is disposed again next to the first wire 51h.

In addition, the angle θ1 of the first wires 51 forming the first helical portion 50 with respect to the axial center C of the catheter 10 is preferably 100 to 140° and more preferably 110 to 130°.

On the other hand, in the second helical portion 60, while being inclined at the predetermined angle θ2 with respect to the axial center C of the catheter 10 as illustrated in FIG. 3, the second wires 61 are helically wound and formed in the direction different from the first helix direction R1, here, counterclockwise (counterclockwise) in the direction opposite to the first belix direction R1 and formed as illustrated in FIG. 2 when the reinforcement member 40 is seen from the base end side to the tip end side of the catheter 10.

The second helical portion 60 includes a parallel contact portion 65 where at least two second wires 61 are disposed in parallel to be in contact with each other, and the parallel contact portions 65 and 65 are wound and formed at a predetermined interval.

In the present embodiment, as illustrated in FIG. 3, the parallel contact portion 65 is configured by disposing two second wires 61 and 61 in parallel to be in contact with each other (disposing the second wires 61 and 61 in parallel to abut against each other without an interval).

More specifically, one edge of one second wire 61 in the width direction (in FIG. 3, the right edge in the second wire 61 disposed on the left side among the two second wires 61 and 61 forming the parallel contact portion 65) and another edge of another second wire 61 in the width direction that is disposed adjacent to the second wire 61 in the axial direction of the second helical portion 60 (in FIG. 3, the left edge in the second wire 61 disposed on the right side among the two second wires 61 and 61 forming the parallel contact portion 65) are disposed to be in contact with each other such that the two second wire 61 are bundled into one set to form the parallel contact portion 65.

In addition, the two parallel contact portions 65 and 65 as one set are disposed at a predetermined interval S2 (refer to FIG. 3) in the axial direction C of the second helical portion 60. This interval S2 on the base end side is wider than that on the tip end side of the second helical portion 60. For convenience of description, FIG. 3 illustrates an intermediate portion of the second helical portion 60. In this intermediate portion, the interval S2 between the second wires 61 and 61 is substantially fixed. The interval S2 on the base end side (in FIG. 3, the right side) with respect to the intermediate portion illustrated in FIG. 3 is wider than the interval S2 on the tip end side (in FIG. 3, the left side) with respect to the intermediate portion illustrated in FIG. 3.

The interval S2 is a length between a tangent line in one edge of one predetermined parallel contact portion 65 in the width direction (in FIG. 3, an edge of the right second wire 61 forming the predetermined parallel contact portion 65) and a tangent line in another edge of another parallel contact portion 65 in the width direction that is disposed adjacent to the predetermined parallel contact portion 65 in the axial direction of the second helical portion 60 (in FIG. 3, an edge of the left second wire 61 forming the other parallel contact portion 65).

In addition, the interval S2 between the parallel contact portions 65 and 65 of the second helical portion 60 is preferably 0.06 to 0.50 mm and more preferably 0.12 to 0.40 mm.

Further, the second helical portion 60 according to the present embodiment is made from eight second wires 61a, 61b, 61c, 61d, 61e, 61f, 61g, and 61h, the second wire 61a and the second wire 61b are in contact with each other, the second wire 61c and the second wire 61d are in contact with each other, the second wire 61e and the second wire 61f are in contact with each other, and the second wire 61g and the second wire 61h are in contact with each other such that two second wires form one parallel contact portion 65, and the eight second wires 61a, 61b, 61c, 61d, 61e, 61f, 61g, and 61h are wound and formed while maintaining the predetermined interval S2 in the axial direction of the second helical portion 60.

Here, the second wires 61a, 61b, 61c, 61d, 61e, 61f, 61g, and 61h are sequentially disposed from the tip end side to the base end side of the second helical portion 60, and one parallel contact portion 65 is configured with the above-described combination. The parallel contact portion 65 made from the second wire 61a and 61b is disposed again next to the parallel contact portion 65 made from the second wire 61g and 61h.

In addition, as illustrated in FIG. 3, in the present embodiment, the parallel contact portion 65 made from two second wires 61 and 61 in contact with each other is knitted to intersect with the first wires 51 of the first helical portion 50 such that the parallel contact portion 65 is positioned alternately outside and inside the first wire 51, for example, is positioned outside (radially outside) the first wire 51 at predetermined two positions in a traveling direction of the second helix direction R2, is positioned inside (radially inside) the first wire 51 at the next two positions, is positioned outside the first wire 51 again at the next two positions, and is subsequently positioned inside, outside, and inside the first wire 51. As a result, the second helical portion 60 is configured.

Further, in the present embodiment, the interval S2 between the parallel contact portions 65 and 65 of the second helical portion 60 is set to be wider than the interval S1 between the first wires 51 and 51 of the first helical portion 50.

In addition, as illustrated in FIG. 3, a space K1 formed by disposing the plurality of first wires 51 of the first helical portion 50 and the plurality of second wires 61 of the second helical portion 60 to intersect with each other is formed in a substantially parallelogram shape (a region that is surrounded by the parallel contact portions 65 and 65 disposed on a short side and the first wires 51 and 51 disposed on a long side), and the space K1 formed in the substantially parallelogram shape is disposed across the entire region of the base end from the tip end of the reinforcement member 40.

Each of the spaces K1 is configured such that the tip end side has a narrower area than the base end side of the reinforcement member 40. For convenience of description, FIG. 3 illustrates an intermediate portion of the reinforcement member 40, and the spaces K1 in the intermediate portion have substantially the same area. However, the area of each of the spaces K1 on the tip end side (in FIG. 3, the left side) with respect to the portion illustrated in FIG. 3 is narrower than the area of each of the spaces K1 on the base end side (in FIG. 3, the right side) with respect to the intermediate portion illustrated in FIG. 3.

Further, in the present embodiment, the number of the first wires 51 forming the first helical portion 50 is the same as the number of the second wires 61 forming the second helical portion 60.

As described above, in the present embodiment, the first wires 51 forming the first helical portion 50 are the eight first wires 51a, 51b, 51c, 51d, 51e, 51f, 51g, and 51h, the second wires 61 forming the second helical portion 60 are the eight second wires 61a, 61b, 61c, 61d, 61e, 61f, 61g, and 61h, and the numbers thereof are the same.

In addition, as illustrated in FIG. 2, in the catheter 10, at least the parallel contact portion 65 is disposed to be in contact with an outer periphery of the inner layer 20, and the outer layer 30 is configured not to penetrate between the outer periphery of the inner layer 20 and the second wires 61 and 61 forming the parallel contact portion 65 that is disposed to be in contact with the outer periphery.

In the present embodiment, the parallel contact portion 65 made from the two second wires 61 and 61 is disposed to be in contact with the outer periphery of the inner layer 20. As a result, a predetermined gap K2 is defined between the outer periphery of the inner layer 20 and the second wires 61 and 61 forming the parallel contact portion 65, and the outer layer 30 is configured not to penetrate into the gap K2. Further, the first wires 51 are disposed to be in contact with the outside (radially outside) the two second wires 61 and 61.

In addition, as illustrated in FIG. 2, the outer layer 30 penetrates into the outside the second wires 61 and 61 forming the parallel contact portion 65 or the first wires 51, and the outer layer 30 also penetrates into the space K1 where the wires 51 and 61 intersect with each other. It can be said that the second wires 61 and 61 or the first wires 51 are buried in the outer layer 30. As a result, displacement (displacement to the radially outside or displacement in a peripheral direction) the second wires 61 and 61 or the first wires 51 with respect to the inner layer 20 is regulated.

In the present embodiment, as described above, the parallel contact portion 65 bas the knitting pattern where the parallel contact portion 65 intersects with the first wires 51 to be positioned alternately outside and inside the first wires 51 (refer to paragraph 0046). Therefore, the parallel contact portion 65 bas a portion in contact with the outer periphery of the inner layer 20 and a portion not in contact with the outer periphery of the inner layer 20 in a helical trajectory in the second belix direction R2 of the second helical portion 60. That is, the entirety of the parallel contact portion 65 does not abut against the outer periphery of the inner layer 20, and the abutting portion and the non-abutting portion are alternately positioned along the helical trajectory of the second helix direction R2.

In addition, as illustrated in FIG. 1, the reinforcement member 40 is not disposed up to the foremost tip end of the inner layer 20, and is disposed in a range from a predetermined position of the inner layer 20 to the front of the foremost tip end thereof. That is, a structure where the reinforcement member 40 is not disposed in the foremost tip end portion of the outer layer 30 is adopted. As a result, the flexibility in the foremost tip end portion of the catheter 10 is ensured.

In addition, the angle θ2 of the second wires 61 forming the second helical portion 60 with respect to the axial center C of the catheter 10 is preferably 100 to 140° and more preferably 110 to 130°.

As the first wires 51 of the first helical portion 50 or the second wires 61 of the second helical portion 60 forming the reinforcement member 40 described above, for example, stainless steel, a piano wire, or a super-elastic alloy such as an Ni—Ti-based alloy, an Ni—Ti—X (X=Fe, Cu, V, Co, Cr, Mn, Nb, or the like) alloy, or a Cu—Zn—X (X=Al, Fe, or the like) alloy can be used. Alternatively, one of radiopaque metals made of W, Pt, Ti, Pd, Rh, Au, Ag, Bi, Ta, an alloy thereof, and the like can also be used. In the present embodiment, the first wires 51 and the second wires 61 are made of W.

In addition, wire diameters of the first wires 51 and the second wires 61 are preferably 0.010 to 0.050 mm and more preferably 0.015 to 0.030 mm. Further, an outer diameter of the reinforcement member 40 is preferably 0.40 to 5.00 mm and more preferably 0.50 to 1.00 mm.

### Modification Example

The shape, structure, material, layout, and the like of each of the members such as the tube, the inner layer, the outer layer, the reinforcement member, the first helical portion, and the second helical portion forming the catheter described above are not limited to the above-described aspect.

In the present embodiment, the inner layer 20 has the shape including the tapered portion 23. However, the inner layer may have a shape that extends with a fixed diameter from the base end to the tip end, may have a shape where a plurality of cylindrical portions extending with a fixed diameter and having different outer diameters are continuously provided in the axial direction (a shape whose outer periphery is stepwise), or may have a shape where a cylindrical portion extending with a fixed diameter and a tapered portion are combined.

In addition, in the present embodiment, the outer layer 30 has the shape where the layers 31, 32, 33, 34, and 35 having different hardnesses are continuously provided in the axial direction. However, the outer layer may include a single layer made of the same material from the base end to the tip end.

Further, in the present embodiment, the first helix direction R1 of the first helical portion 50 forming the reinforcement member 40 is clockwise, and the second helix direction R2 of the second helical portion 60 is counterclockwise. For example, in a state where both of the first helix direction RI of the first helical portion and the second helix direction R2 of the second helical portion are clockwise or counterclockwise, the inclination angle θ1 of the first wires 51 with respect to the axial center C of the catheter 10 and inclination angle θ2 of the second wires 61 with respect to the axial center C of the catheter 10 may be different, as long as the first helix direction RI and the second helix direction R2 are different.

In addition, in the present embodiment, by disposing the two second wires 61 in parallel to be in contact with each other, the parallel contact portion 65 is configured. However, the number of the second wires 61 forming the parallel contact portion may be three or more.

Further, in the present embodiment, the interval S2 between the parallel contact portions 65 and 65 of the second helical portion 60 is set to be wider than the interval S1 between the first wires 51 and 51 of the first helical portion 50. However, the interval S2 may narrower than the interval S1, or both of the intervals S1 and S2 may be the same.

In addition, the number of the first wires 51 forming the first helical portion 50 is the same as the number of the second wires 61 forming the second helical portion 60. However, the number of the first wires and the number of the second wires may be different, or the number of either one of the first wires or the second wires may be more than or less than the number of the other one.

Further, in the present embodiment, the knitting pattern of the parallel contact portion 65 with respect to the first wires 51 is formed such that the parallel contact portion 65 alternately intersects with the outside and the inside of the first wires 51 for every two positions in the traveling direction of the second helix direction R2 (refer to FIG. 3). However, for example, a knitting pattern where the parallel contact portion 65 alternately intersects with the outside and the inside of the first wires for every position may be adopted.

In addition, in the present embodiment, the interval S1 between the first wires 51 and 51 of the first helical portion 50 on the base end side of the first helical portion 50 is wider than that on the tip end side. However, the interval S1 may be fixed in the entire region in the axial direction from the base end of the first helical portion 50 to the tip end thereof, the tip end side of the first helical portion 50 may be wider than the base end side, may be gradually widened from the base end of the first helical portion 50 to the tip end thereof, or may be gradually narrowed from the base end of the first helical portion 50 to the tip end thereof.

Further, in the present embodiment, the interval S2 between the parallel contact portions 65 and 65 of the second helical portion 60 on the base end side of the second helical portion 60 is wider than that on the tip end side However, the interval S2 may be fixed in the entire region in the axial direction from the base end of the second helical portion 60 to the tip end thereof, the tip end side of the second helical portion 60 may be wider than the base end side, may be gradually widened from the base end of the second helical portion 60 to the tip end thereof, or may be gradually narrowed from the base end of the second helical portion 60 to the tip end thereof.

### Effects

Next, an example of a method of using the catheter according to the present invention made from the above-described structure, effects thereof, and the like will be described.

The catheter 10 is used for injecting a contrast medium, an anticancer drug or the like or inserting a stent or the like into, for example, a tubular organ of the human body such as a blood vessel of hepatic artery or the like, a bile duct, a pancreatic duct, a ureter, or a trachea or into a predetermined position of a human tissue such as a body cavity. The catheter 10 may be used for, for example, a position other than the above-described positions, and the usage position is not particularly limited.

First, after administering a contrast medium into the body, the catheter 10 is moved while being guided through a guide wire (not illustrated) using the well-known Seldinger method or the like under X-ray illumination (radioscopy).

At this time, it may be desired to allow the catheter 10 to travel through a predetermined small-diameter tubular organ selected from a plurality of small-diameter tubular organs branched from a large-diameter tubular organ. To that end, flexibility is required for the catheter 10. In addition, in order to allow a drug solution such as an anticancer drug to reliably reach an affected part, it is required that the tube inner cavity of the catheter 10 can be maintained without a kink, that is, kink resistance is required.

On the other hand, in the catheter 10 according to the present invention, as described above, the second helical portion 60 includes the parallel contact portion 65 where at least two second wires 61 and 61 are disposed in parallel to be in contact with each other, and the parallel contact portions 65 and 65 are wound and formed at the predetermined interval.

Therefore, in the parallel contact portion 65, while maintaining the kink resistance of the catheter 10, the flexibility of the catheter 10 can be improved with the interval between the parallel contact portions 65 and 65.

Accordingly, the above-described requirements (the maintenance of the kink resistance and the improvement of the flexibility) can be satisfied. Accordingly, for example, during treatment for liver cancer, the catheter 10 can be suitably used, for example, when being inserted into hepatic artery to inject an anticancer drug into the liver through the hepatic artery.

Incidentally, in order to maintain the kink resistance of the catheter 10, for example, it is also considered to use one thick wire as the second wire 61 forming the second helical portion 60. However, in this case, the kink resistance of the catheter 10 can be maintained, but the flexibility of the catheter 10 is likely to decrease.

On the other hand, the catheter 10 according to the present invention includes the parallel contact portion 65 where at least two second wires 61 forming the second helical portion 60 are disposed in parallel to be in contact with each other. Therefore, the same kink resistance as that of a case where one thick wire is used as a bundle of a plurality of wires can be obtained, a thin wire that is more flexible than a thick line can be used while maintaining kink resistance, and the flexibility of the catheter 10 can be improved with the interval S2 between the parallel contact portions 65 and 65.

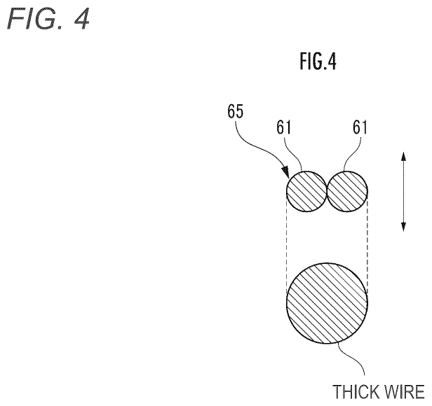

Further, when at least two second wires 61 and 61 are disposed in parallel to be in contact with each other, an increase in size in the outer diameter direction can be further suppressed as compared to one thick wire. That is, as illustrated in FIG. 4, as compared to one thick wire having an outer diameter that is the same as the dimension of the width (the horizontal direction on the plane of FIG. 4) of the parallel contact portion 65 where two second wires 61 and 61 are disposed to be in contact with each other, the dimension of the two second wires 61 and 61 in the outer diameter direction (which refers to the dimension in the vertical direction on the paper plane of FIG. 4; refer to an arrow of FIG. 4) can be set to be less than the outer diameter of the one thick wire. As a result, the outer diameter of the catheter 10 can be reduced.

In addition, in the present embodiment, the interval S2 between the parallel contact portions 65 and 65 of the second helical portion 60 is set to be wider than the interval SI between the first wires 51 and 51 of the first helical portion 50.

According to the above-described aspect, the interval S2 is set as described above. Therefore, the space K1 where the second wires 61 of the second helical portion 60 are formed to intersect with the first wires 51 of the first helical portion 50 can be ensured to be wider, and the flexibility of the catheter 10 can be further improved. In addition, the number of the second wires 61 of the second helical portion 60 in a predetermined region can be approximated to the number of the first wires 51 of the first helical portion 50. From this viewpoint, the flexibility of the catheter 10 can also be improved.

Further, in the present embodiment, the number of the first wires 51 forming the first helical portion 50 is the same as the number of the second wires 61 forming the second helical portion 60.

In the above-described aspect, the number of the first wires 51 forming the first helical portion 50 is the same as the number of the second wires 61 forming the second helical portion 60. Therefore, the interval between the parallel contact portions 65 and 65 in the second helical portion 60 is likely to be widened, and the flexibility of the catheter 10 can be further improved.

In addition, in the present embodiment, the tube includes the inner layer 20 and the outer layer 30 that is disposed outside the inner layer 20, at least the parallel contact portion 65 is disposed to be in contact with the outer periphery of the inner layer 20, and the outer layer 30 is configured not to penetrate between the outer periphery of the inner layer 20 and the second wires 61 and 61 forming the parallel contact portion 65 that is disposed to be in contact with the outer periphery (refer to FIG. 2).

According to the above-described aspect, the outer layer 30 is configured not to penetrate between the outer periphery of the inner layer 20 and the second wires 61 and 61 forming the parallel contact portion 65 that is disposed to be in contact with the outer periphery, and thus the second wires 61 and 61 forming the parallel contact portion 65 can be displaced to some extent without being completely restricted. Therefore, the flexibility of the reinforcement member 40 can be improved, and further the flexibility of the catheter 10 can be improved.

### EXAMPLE

### Example

A catheter according to Example having the same shape and structure as those of FIGS. 1 to 3 was manufactured. The first and second helical portions 50 and 60 were formed by winding eight first wires 51 and eight second wires 61, respectively.

### Comparative Example

A catheter according to Comparative Example was manufactured, in which the first and second helical portions were formed by winding eight first wires and eight second wires, respectively, the inclination angles θ1 and θ2 of the first and second wires with respect to the catheter axial center were set to be the same as those of Example, and the second helical portion where the second wires were wound at an interval in the axial direction without being in contact with each other was provided.

### Flexibility Check Test

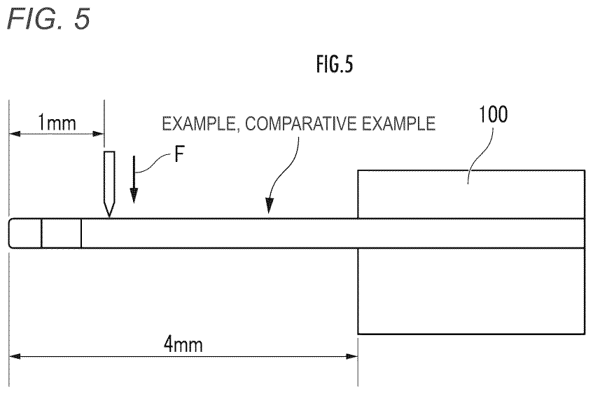

As illustrated in FIG. 5, each of the catheters according to Example and Comparative Example was set to a well-known indentation load measurement tester 100. Each of the catheters was fixed such that the foremost tip end thereof was fixed at a position of 4 mm from an end surface of the indentation load measurement tester 100. At a position of 1 mm from the tip end of each of the catheter, an indentation load F was applied at a predetermined speed to form an indentation of 0.5 mm. At this time, a repulsive load (resistance load) was measured. In addition, this test was performed three times on each of the catheters according to Example and Comparative Example.

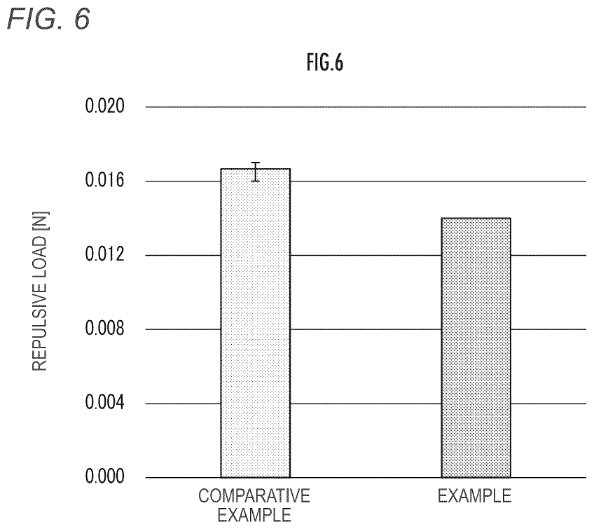

The results are illustrated in FIG. 6. FIG. 6 illustrates that, when the repulsive load is low, the flexibility is high. As illustrated in FIG. 6, it can be seen that the repulsive load of Example is lower than the repulsive load of Comparative Example, and the flexibility of Example is higher than the flexibility of Comparative Example.

### Kink Resistance Test

Using a predetermined measurement tool where an object was able to be sandwiched, the tip end portion of each of the catheters according to Example and Comparative Example was sandwiched to form a U-shape. Next, the sandwiching amount by the measurement tool was gradually decreased, and when the sandwiching amount reached a predetermined value, the outer diameter was measured. In addition, this test was performed three times on each of the catheters according to Example and Comparative Example.

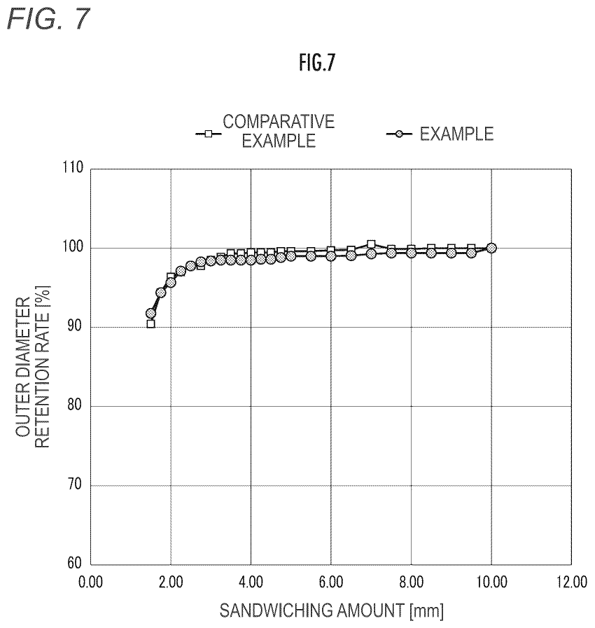

The results are illustrated in FIG. 7. The horizontal axis in FIG. 7 represents the sandwiching amount of each of the catheters by the measurement tool. In addition, the vertical axis in FIG. 7 represents an outer diameter retention rate, which shows a percentage with respect to the outer diameter of the catheter at the start of the test (at an initial stage) (the outer diameter at the initial stage/the outer diameter at the time of the measurement×100). It can be said that, when the outer diameter retention rate decreases, a kink is generated.

As illustrated in FIG. 7, the catheter according to Example and the catheter according to Comparative Example had the same tendency of a decrease in the outer diameter retention rate. Accordingly, it can be seen that the catheter according to Example and the catheter according to Comparative Example had substantially the same kink resistance.

The present invention is not limited to the above-described embodiment various modification embodiments can be made within the scope of gist of the present invention, and these embodiments are also included in the range of the present invention.

### REFERENCE SIGNS LIST

10: catheter
20: inner layer
30: outer layer
40: reinforcement member
50: first helical portion
51: first wire
60: second helical portion
61: second wire
65: parallel contact portion

1. A catheter comprising a tube that includes a reinforcement member made from a braid, wherein
the reinforcement member includes
a first helical portion made from a plurality of first wires, and formed by winding the plurality of first wires at a predetermined interval in a first helix direction, and
a second helical portion formed by winding second wires in a second helix direction different from the first helix direction,
the second helical portion includes a parallel contact portion where at least two of the second wires are disposed in parallel to be in contact with each other, and the parallel contact portion is knitted and formed to intersect with the first wires such that the parallel contact portion is positioned outside and inside of the first wires, and
the parallel contact portion includes at least two sets of one set where at least two of the second wires are disposed in parallel to be in contact with each other and another set where at least two of the second wires different from the second wires forming the one set are disposed in parallel to be in contact with each other, and the second helical portion is formed by winding the two sets at a predetermined interval.

2. The catheter according to claim 1, wherein the interval between the two sets in the parallel contact portion of the second helical portion is set to be wider than the interval between the plurality of first wires of the first helical portion.

3. The catheter according to claim 1, wherein the number of the first wires forming the first helical portion is the same as the number of the second wires forming the second helical portion.

4. The catheter according to claim 1, wherein
the tube includes an inner layer and an outer layer that is disposed outside the inner layer,
at least the parallel contact portion is disposed to be in contact with an outer periphery of the inner layer, and
the outer layer is configured not to penetrate between the outer periphery of the inner layer and the second wires forming the parallel contact portion that is disposed to be in contact with the outer periphery.
